(19) United States
(12) Patent Application Publication
(10) Pub. No.: US 2008/0249153 A1
(43) Pub. Date: Oct. 9, 2008

(54) ANTHELMINTIC FORMULATIONS

(76) Inventor: Majid Hameed Abdul Razzak, Albany (NZ)

Correspondence Address: FROMMER LAWRENCE & HAUG, 745 FIFTH AVENUE- 10TH FL., NEW YORK, NY, 10151, US

(21) Appl. No.: 12/049,039

(22) Filed: Mar. 14, 2008

Related U.S. Application Data
(63) Continuation-in-part of application No. PCT/NZ2005/000243, filed on Sep. 15, 2005.

(51) Int. Cl. A61K 31/4184; A61P 33/10

(52) U.S. Cl. 514/388

(57) ABSTRACT

This invention relates to the preparation of veterinary anthelmintic formulations including triclabendazole in solution, particularly for the treatment of parasitic infections such as fasciolisis, and particularly for administration to an animal in the form of a pour-on. The solutions may include triclabendazole dissolved in a solvent system including at least one solvent selected from 2-pyrrolidone and liquid polyethylene glycol. Additional solvents may also be included. The present invention is advantageous as triclabendazole can be included in solution up to a concentration of about 60% w/v allowing for an effective dose to be delivered to the animal in a volume of 25 ml or less.

### INCORPORATION BY REFERENCE

This application is a continuation-in-part application of international patent application Serial No. PCT/NZ2005/000243 filed 15 Sep. 2005, which published as PCT Publication No. WO 2007/032688 on 22 Mar. 2007.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to liquid solutions of triclabendazole and in particular to veterinary anthelmintic formulations including triclabendazole in liquid solution, it has particular, though no sole, application to liquid pour-on formulations.

### BACKGROUND OF THE INVENTION

Diseases caused by parasitic helminths such as nematodes, cestodes and trematodes (liver fluke) can cause severe economic losses in the farming of ruminants and other animals. Fasciolisis, a disease caused by parasitic liver flukes, is commonly contracted by domestic herbivorous animals such as sheep, cattle and goats. The liver flukes Fasciola hepatica and Fasciola gigantica are flat worms which live in the bile ducts (in the liver) of their hosts.

Fasciola hepatica infection is widespread and is generally associated with low lying wet or water-covered areas. Areas where the average annual rainfall is at or above about 600 mm and irrigation areas in particular, tend to create ideal living/propagating environments for aquatic snails which serve as intermediate hosts for first larvae of fasciola hepatica (miracidia). The miracidia develop and multiply and eventually leave the snail host and encyst on vegetation forming metacercarial (infective stage of fasciola hepatica). When the vegetation is consumed by a grazing animal, the metacercarial excysts in the small intestine releasing the young parasite. These immature flukes penetrate the intestinal wall and enter the abdominal cavity. From there they migrate to the liver. Acute infections can result from the immature flukes burrowing through the liver substance. Death often follows as a result of concomitant blood loss.

It has been estimated that around the world at least 40 million sheep and 6 million cattle graze on pastures contaminated with fasciola hepatica (liver fluke).

Because ruminants, such as sheep, cattle and goats, are susceptible to fasciolisis, it is of critical importance that formulations effective against the disease are available.

Until recently, the treatment of fasciolisis has been less than optimal. The drug previously used, bithionol, had to be administered orally over five days at a dosage of 30 mg/kg of body weight. Praziquantel, a very effective drug against most trematode infections, is inactive against Fasciola species.

The benzimidazole class of active agents are known for their anthelmintic activity. They are known to be sparingly soluble and are either made up in tablet or powder form (for use with small animals) or typically made up as suspensions for use in oral drenches.

Of all the benzimidazoles known, triclabendazole, which is a halogenated benzimidazole, is highly effective against liver flukes (fasciolisis) at all stages of their life cycle. Triclabendazole is known as 5-Chloro-6-(3,3-dichlorophenoxy)-2-methylthio-1H-benzimidazole, and is represented by the following structural formula:

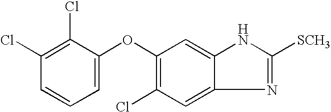

Other active agents within the benzimidazole class of actives such as albendazole are only effective against adult fluke.

In general for ease of application to large numbers of animals, farmers and veterinary surgeons prefer to treat farm animals with pour-ons rather than oral drenches or injectables.

Pour-ons are typically relatively viscous liquids applied in small doses to the neck or back line of the animal. In the case of sheep pour on, the applicator guns are adapted to supply a dose of about 5 ml to 10 ml, and in the case of cattle the dose (depending upon the product) is typically about 40 ml to 60 ml. Most pour-ons have the active in solution, and the solvent or solvent system is chosen to allow the active to pass through the dermal layer and provide a sufficiently high systemic amount of the active quickly enough.

The solvent system needs to be non-irritant to the farmer and the animal, stable, non-toxic, non-carcinogenic, and capable of being used in a pour-on applicator gun. Because of the small volume of each dose (too much would result in liquid running off the animal's back) the pour-on formulation is typically designed so that sufficient active is contained within the solvent system that a typical designed dose rate of 1 ml of pour-on for each 10 kg of animal live weight is standard. Thus a 10 ml dose of a pour-on would normally supply sufficient active to treat a 100 kg sheep, 50 ml would supply normally enough active to treat a 500 kg cattle beast.

Parasitologists on the other hand recommend the use of oral drenches as there is usually a better take up of active from an oral drench than a pour-on.

Formulators prefer to design solutions rather than suspensions for veterinary purposes, as solutions can normally be used for either the pour-on route of administration or the oral route of administration, and solutions are usually far more stable than suspensions when stored in bulk. However, if the active is known to be sparingly soluble in water or most practical solvents then the formulator will favour a suspension for use as an oral drench.

It is particularly advantageous to provide liquid formulations which contain a sufficient quantity of an active anthelmintic agent in a solution which can be easily administered by way of a pour-on.

It has been difficult to provide liquid formulations containing triclabendazole in solution. Indeed, until recently it has only been available as a suspension formulation for oral administration.

Commercial drench formulations of triclabendazole are known in which triclabendazole is suspended in a liquid carrier. Such formulations are administered orally via an appropriate drenching apparatus or by subcutaneous injection. Oral triclabendazole suspension formulations are commercially available and marketed under the trade name ‘Fasinex’120 or 240. Such formulations can have a triclabendazole concentration in the order of 120 g/l (or 240 g/l), which equates to 12% w/v (24% w/v). A 50 ml (25 ml) dose of Fasinex 120 (240) equates to a dose of 6 g triclabendazole/500 kg beast (12 mg/kg).

While oral (drench) suspension formulations containing triclabendazole have been available and successful in treating fasciolisis, the method of administering a suitable volume of oral formulation often requires a suitably experienced/qualified person to administer a dose. Dosing a herd of animals can therefore be laborious, time consuming and place a severe economic strain on a conventional farm. Treatment of fasciolisis by way of a single injection represents an even less practical option for a farmer.

Pour-on formulations containing a benzimidazole compound are therefore desirable within the farming community but attempts to produce pour ons containing benzimidazoles have been unsuccessful until the applicant invented a formulation for a triclabendazole pour on. The applicant had previously tried unsuccessfully to formulate an oxfendazole pour on.

In WO95/23590 (Bomac), a pour-on formulation is described in which a benzimidazole, selected from oxfendazole and/or albendazole, is formulated in a suspension. Suspensions of albendazole in accordance with formulations described in ‘Bomac’ have not been effective in the treatment of a parasitic burden. In any case as stated earlier albendazole is not the preferred active for treatment of liver fluke.

Other pour-on formulations have been suggested in patent literature in which an active agent is dissolved; emulsified; or suspended in a solvent/solvent mixture. Anthelmintic formulations are known which contain oxfendazole; tetramisole and levamisole that exhibit anthelmintic action as a pour-on comparable to that of analogous oral or injectable treatments. French patent registration no. 96 14068 teaches a formulation for topical administration including oxfendazole in an amount of 5% w/v dissolved in a non-aqueous ‘vehicle’, a non-aqueous co-solvent, a non-ionic surfactant and a polymer. As stated earlier, however, such anthelmintics are not effective in the treatment of early immature and immature fasciola hepatica.

The solubility characteristic of triclabendazole makes formulating an effective triclabendazole pour-on extremely difficult. A number of rate limiting barriers are faced in formulating active ingredient(s) in a commercially effective dosage form including (i) efficient and sufficient absorption to provide systemic amounts of active quickly; (ii) minimal exposure of an animal to a toxic dosage form; and (iii) stability of formulation.

These and other limitations are amplified given the characteristics of triclabendazole and the desire to provide a triclabendazole pour-on formulation, which is additionally required to permeate across an animal's dermis, and be sufficiently absorbed and transported to the site of infection (liver). Even further barriers exist to the extent that a sufficient kill rate of liver fluke needs to be achieved to substantially minimise potential development of drug resistant parasitic strains.

For triclabendazole pour-on an effective dose is a dose of about 30 mg per kg animal weight. If the triclabendazole is present in solution at 150 mg per ml or 15% w/v, a dose of 70 ml will deliver sufficient triclabendazole to treat fasciolisis in a cow weighing 350 kg.

PCT/NZ00/00053 discloses certain solvents which are capable of dissolving triclabendazole. The specific solvents disclosed are benzyl alcohol, n-methyl pyrrolidone, glycol ethers including butyl dioxitol. The formulations included as examples incorporated triclabendazole at 30% w/v. Thus a dose of 35 ml will deliver sufficient triclabendazole to treat fascioliasis in a cow weighing 350 kg.

While this is a significant advance it would be useful to be able to include triclabendazole in solution with other solvents for pour-ons and for other routes of administration, and in the case of a pour on formulation preferably containing higher concentrations of triclabendazole to allow an even smaller volume of formulation to be applied to the animal while still providing an effective dose.

While reference has been made to prior art in the specification it is not to be taken as an admission that the cited art forms part of the common general knowledge.

### OBJECT OF THE INVENTION

It is an object of the invention to provide an improved veterinary anthelmintic formulation including triclabendazole in solution, or one which will at least provide the public with a useful choice.

### SUMMARY OF THE INVENTION

In one aspect the invention provides a veterinary anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.

Preferably the solvent system may also include one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.

In its most preferred aspect the invention provides a stable liquid veterinary pour-on anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.

Preferably the solvent system may also include one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.

Preferably the solvent system may also contain 2-pyrrolidone and at least one additional solvent selected from the group comprising butyl dioxitol, N-methyl pyrrolidone, benzyl alcohol, glycerol formal, propylene glycol and ethyl alcohol.

Preferably the triclabendazole may be present in the range of 5-60% w/v.

Preferably the triclabendazole may be present in a sufficiently high concentration such that an effective amount of triclabendazole may be delivered to an animal in a volume of substantially 25 ml or less.

Preferably the triclabendazole may be present in the range of 40-60% w/v.

Preferably the formulation may include at least one additional anthelmintic active or other medicament which is soluble in the solvent system.

Preferably the additional anthelmintic active or actives may be selected from the group comprising avermectins, milbemycins, tetramisole and levamisole.

Preferably the additional anthelmintic active may be an avermectin, such as abamectin.

Preferably the formulation may further include excipients such as dyes, preservatives, stabilisers, buffers, thickeners, anti-foaming agents, spreading agents and the like.

Preferably the formulation may contain (a) about 60% w/v of triclabendazole, (b) about 1.0% w/v of abamectin, and a solvent system, wherein the solvent system includes N-methyl pyrrolidone in an amount of about 10% w/v and 2-pyrrolidone in an amount of about 29% w/v.

In another aspect the invention provides a method of treating parasites including fasciolisis in warm blooded animals by administering to an animal a formulation as described above.

In another aspect the invention provides a method of treating parasites including fasciolisis in warm blooded animals by providing a pour on formulation as described above and administering a volume of about 25 ml or less of the pour-on formulation on to the skin surface of an animal.

Preferably the method of treating fasciolisis in warm blooded animals may involve administering a volume of the pour on capable of delivering at least 1 mg of triclabendazole per 10 kg of body weight of the animal to be treated.

References to ‘medicaments’ include those substances such as anthelmintics, antigens, vaccines, vitamin and mineral supplements and other substances, which may be useful for promoting the health of the animal.

‘Liquid polyethylene glycol’ may include polyethylene glycol (PEG) that is liquid at room temperature. PEG 200 to 400 are liquid at room temperature. In addition, blends containing polyethylene glycols of higher or lower molecular weight are useable if the resulting mixture is liquid at room temperature.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

Benzimidazoles are generally regarded as being difficult to dissolve, and consequently commercial formulations are made up as liquid suspensions mainly for application as oral drenches.

As a comparison trials were conducted to determine the solubility of various members of the benzimidazole family in different solvents. It was found that oxfendazole, albendazole and ricobendazole are all either insoluble or so insignificantly soluble that they cannot be formulated as effective veterinary solutions in solvents such as benzyl alcohol, propylene glycol, NMP, 2-pyrollidone, PEG, glycerol formal and butyl dioxitol.

When trials were conducted with triclabendazole it was found that the triclabendazole was more soluble generally in the solvents mentioned above than the other benzimidazoles which were sparingly soluble at best, and in most cases insoluble. But more importantly it was discovered that triclabendazole is extremely soluble in 2-pyrrolidone and PEG 400 with more than 500 mg of triclabendazole capable of being dissolved in a ml of these solvents. This is particularly advantageous as it allows for formulations of low volume to be formulated for delivery to cattle and other animals.

TABLE 1a
Solubility Studies of triclabendazole in different organic solvents

| | | Quantity | | | Amount |
|---|---|---|---|---|---|
| Sample | | dissolved in | Total Volume | | dissolved |
| No. | Solvent Name | 10 ml solvent | occupied | | (mg/ml) |
| 1 | Benzyl Alcohol | 5.0 g (Max | 14 | ml | 357.14 |
| | | solubility) | | | |
| 2 | Propylene | 1.5 g (Max | 11.0 | ml | 136.36 |
| | Glycol | solubility) | | | |
| 3 | NMP^{α} | 4.5 g (Max | 14.0 | ml | 321.43 |
| | | solubility) | | | |
| 4 | 2-pyrrolidone | 10.0 g (flowable | 17.5-18.0 | ml | 555.55 |
| | | in nature) | | | |
| 5 | PEG400 | 10.0 g (flowable | 17.5 | ml | 571.43 |
| | | in nature) | | | |
| 6 | Glyceryl | 1 g (Max | | | 90.91 |
| | Formal | solubility) | | | |
| 7 | DGBE (butyl | 2.0 g (Max | | | 166.66 |
| | dioxitol) | solubility) | | | |

^{α}NMP—N-methyl pyrrolidone

TABLE 1b
Solubility studies of triclabendazole in different alcohols

| | | | | | Solvent | Solvent |
|---|---|---|---|---|---|---|
| | | | | | added to | added to |
| | | Triclabendazole | Triclabendazole | Triclabendazole | the | the |
| | | taken in 10 ml | added to | added to | previous | previous |
| Sample | Solvent | solvent. | previous | previous | system. | system. |
| No. | Name | Day 0 | system. Day 1 | system. Day 7 | Day 12 | Day 15 |
| 1 | Methyl | 500 mg | 500 mg | 1 g | 5 ml | 5 ml |
| | alcohol | | | | | |
| 2 | Ethyl | 500 mg | 500 mg | 1 g | 5 ml | 5 ml |
| | alcohol | | | | | |
| 3 | Isopropyl | 500 mg | 500 mg | 1 g | 5 ml | — |
| | alcohol | | | | | |
| 4 | 1-butanol | 500 mg | 500 mg | 1 g | 5 ml | — |
| 5 | 1- | 500 mg | 500 mg | 1 g | 5 ml | — |
| | hexanol | | | | | |

TABLE 1c
Solubility Studies of Triclabendazole is various alcohols

| | | Quantity | Total | Amount |
|---|---|---|---|---|
| Sample | Solvent | dissolved in | volume | dissolved |
| No. | Name | solvent) | occupied | (mg/ml) |
| 1 | Methyl | 2 gm/20 ml | 22.5 ml | 88.88 |
| | alcohol | | | |
| 2 | Ethyl | 2 gm/20 ml | 22.5 ml | 88.88 |
| | alcohol | | | |
| 3 | Isopropyl | 2 gm/15 ml | 17.0 ml | 117.5 |
| | alcohol | | | |
| 4 | 1-butanol | 2 gm/15 ml | 17.0 ml | 117.5 |
| 5 | 1-hexanol | 2 gm/15 ml | 17.0 ml | 117.5 |

Based on the above solubility data various high concentration formulations containing 2-pyrrolidone or PEG were proposed. The formulations were prepared and stored at room temperature and refrigeration temperature for 30 days. The formulations showed no deposition at room temperature or under refrigeration.

In field use these formulations would be highly desirable as they could provide the flexibility to deliver high concentrations of triclabendazole to the animal in a relatively small amount of solvent. The ideal would be to deliver concentrated quantities of triclabendazole in a solvent delivered at the rate of as low as 1 ml per 20 kg bodyweight.

After conducting a number of solubility studies of benzimidazoles in general and triclabendazole in particular it has now been found that triclabendazole is extremely soluble in 2-pyrrolidone and liquid polyethylene glycol (particularly PEG 400), with more than 500 mg of triclabendazole dissolving in a millilitre of each of these solvents.

It has been found that stable liquid formulations containing triclabendazole in solution with these solvents can be prepared and that these actives can be absorbed through the skin to control parasitic diseases, including fasciolisis in warm blooded animals. The use of these particular solvents is advantageous as it allows high concentrations of triclabendazole to be dissolved so that doses of low volume can be administered to cattle and other animals.

Based on the solubility studies various formulations containing 2-pyrrolidone or liquid polyethylene glycol were prepared in accordance with the following examples.

The example formulations below are particularly suitable for administration as pour-ons. However, the solutions of the present invention are useable in different dosage forms. By way of example the triclabendazole solution may be incorporated into oral dosage forms as tablets, capsules (each of which includes sustained release or timed release formulations) and drenches.

Other topical administration forms such as spot-ons are useable. The various dosage forms are well known to those of ordinary skill in the pharmaceutical and veterinary arts.

The invention will now be further described by way of the following non-limiting examples.

### EXAMPLES

### Example 1

| Ingredients | Quantity | |
|---|---|---|
| Triclabendazole | 30 | g |
| 2-pyrrolidone | 50 | g |
| DGBE (butyl dioxitol) q.s. to | 100 | ml |

To prepare the formulation add the triclabendazole and 2-pyrrolidone. Warm to 60° C. and stir to dissolve. Cool mix to less than 25° C. and dilute to volume with DGBE.

### Example 2

| Ingredients | Quantity | |
|---|---|---|
| Triclabendazole | 30 | g |
| PEG 400 | 50 | g |
| DGBE (butyl dioxitol) q.s. to | 100 | ml |

To prepare the formulation add the triclabendazole and PEG 400 (polyethylene glycol 400). Warm to 60° C. and stir to dissolve. Cool mix to less than 25° C. and dilute to volume with DGBE.

### Example 3

| Ingredients | Quantity | |
|---|---|---|
| Triclabendazole | 60 | g |
| 2-pyrrolidone q.s. to | 100 | ml |

To prepare the formulation add the triclabendazole, and 2-pyrolidone. Warm to 70° C. and stir to dissolve. Cool mix to less than 25° C. and dilute to volume with 2-pyrrolidone.

### Example 4

| Ingredients | Quantity | |
|---|---|---|
| Triclabendazole | 60 | g |
| N-methyl pyrrolidone | 10 | g |
| 2-pyrolidone q.s. to | 100 | ml |

To prepare the formulation add the triclabendazole, NMP and 2-pyrrolidone. Warm to 70° C. and stir to dissolve. Cool mix to less than 25° C. and dilute to volume with 2-pyrrolidone.

### Example 5

| Ingredients | Quantity | |
|---|---|---|
| Triclabendazole | 60 | g |
| Benzyl Alcohol | 10 | g |
| 2-pyrrolidone q.s. to | 100 | ml |

To prepare the formulation add the triclabendazole, Benzyl Alcohol and 2-pyrrolidone. Warm to 70° C. and stir to dissolve. Cool mix to less than 25° C. and dilute to volume with 2-pyrrolidone.

### Example 6

| Ingredients | Quantity | |
|---|---|---|
| Triclabendazole | 60 | g |
| DGBE | 10 | g |
| 2-pyrolidone q.s. to | 100 | ml |

To prepare the formulation add the triclabendazole, DGBE and 2-pyrrolidone. Warm to 70° C. and stir to dissolve. Cool mix to less than 25° C. and dilute to volume with 2-pyrrolidone.

### Example 7

| Ingredients | Quantity | |
|---|---|---|
| Triclabendazole | 60 | g |
| Glycerol Formal | 10 | g |
| 2-pyrrolidone q.s. to | 100 | ml |

To prepare the formulation add the triclabendazole, Glycerol Formal and 2-pyrrolidone. Warm to 70° C. and stir to dissolve. Cool mix to less than 25° C. and dilute to volume with 2-pyrrolidone.

### Example 8

| Ingredients | Quantity | |
|---|---|---|
| Triclabendazole | 40 | g |
| PEG 400 q.s. to | 100 | ml |

To prepare the formulation add the triclabendazole and PEG 400. Warm to 70° C. and stir to dissolve. Cool mix to less than 25° C. and dilute to volume with PEG 400.

### Example 9

| Ingredients | Quantity | |
|---|---|---|
| Triclabendazole | 40 | g |
| DGBE | 10 | g |
| PEG 400 q.s. to | 100 | ml |

To prepare the formulation add the triclabendazole, DGBE and PEG 400. Warm to 70° C. and stir to dissolve. Cool mix to less than 25° C. and dilute to volume with PEG 400.

### Example 10

| Ingredients | Quantity | |
|---|---|---|
| Triclabendazole | 40 | g |
| 2-pyrrolidone q.s. to | 100 | ml |

To prepare the formulation add the triclabendazole, and 2-pyrrolidone. Warm to 70° C. and stir to dissolve. Cool mix to less than 25° C. and dilute to volume with 2-pyrrolidone.

### Example 11

| S. No. | Ingredients | Quantity | |
|---|---|---|---|
| 1 | Triclabendazole | 40 | g |
| 2 | NMP | 10 | g |
| 3 | 2-pyrrolidone q.s. to | 100 | ml |

To prepare the formulation add the triclabendazole, NMP and 2-pyrrolidone. Warm to 70° C. and stir to dissolve. Cool mix to less than 25° C. and dilute to volume with 2-pyrrolidone.

### Example 12

| S. No. | Ingredients | Quantity | |
|---|---|---|---|
| 1 | Triclabendazole | 40 | g |
| 2 | Benzyl alcohol | 10 | g |
| 3 | 2-pyrrolidone q.s. to | 100 | ml |

To prepare the formulation add the triclabendazole, benzyl alcohol and 2-pyrrolidone. Warm to 70° C. and stir to dissolve. Cool mix to less than 25° C. and dilute to volume with 2-pyrrolidone.

### Example 13

| Ingredients | Quantity | |
|---|---|---|
| Triclabendazole | 40 | g |
| Glycerol formal | 10 | g |
| 2-pyrrolidone q.s. to | 100 | ml |

To prepare the formulation add the triclabendazole, glycerol formal and 2-pyrrolidone. Warm to 70° C. and stir to dissolve. Cool mix to less than 25° C. and dilute to volume with 2-pyrrolidone.

### Example 14

| Ingredients | Quantity | |
|---|---|---|
| Triclabendazole | 40 | g |
| Propylene glycol | 10 | g |
| 2-pyrrolidone q.s. to | 100 | ml |

To prepare the formulation add the triclabendazole, propylene glycol and 2-pyrrolidone. Warm to 70° C. and stir to dissolve. Cool mix to less than 25° C. and dilute to volume with 2-pyrrolidone.

### Example 15

| Ingredients | Quantity | |
|---|---|---|
| Triclabendazole | 20 | g |
| 2-pyrrolidone q.s. to | 100 | ml |

To prepare the formulation add the triclabendazole, and 2-pyrrolidone. Warm to 70° C. and stir to dissolve. Cool mix to less than 25° C. and dilute to volume with 2-pyrrolidone.

### Example 16

| Ingredients | Quantity | |
|---|---|---|
| Triclabendazole | 20 | g |
| NMP | 10 | g |
| 2-pyrrolidone q.s. to | 100 | ml |

To prepare the formulation add the triclabendazole, NMP and 2-pyrrolidone. Warm to 70° C. and stir to dissolve. Cool mix to less than 25° C. and dilute to volume with 2-pyrrolidone.

### Example 17

| Ingredients | Quantity | |
|---|---|---|
| Triclabendazole | 20 | g |
| Benzyl alcohol | 10 | g |
| 2-pyrrolidone q.s. to | 100 | ml |

To prepare the formulation add the triclabendazole, benzyl alcohol and 2-pyrrolidone. Warm to 70° C. and stir to dissolve. Cool mix to less than 25° C. and dilute to volume with 2-pyrrolidone.

### Example 18

| Ingredients | Quantity | |
|---|---|---|
| Triclabendazole | 20 | g |
| Glycerol formal | 10 | g |
| 2-pyrrolidone q.s. to | 100 | ml |

To prepare the formulation add the triclabendazole, glycerol formal and 2-pyrrolidone. Warm to 70° C. and stir to dissolve. Cool mix to less than 25° C. and dilute to volume with 2-pyrrolidone.

### Example 19

| Ingredients | Quantity | |
|---|---|---|
| Triclabendazole | 20 | g |
| Propylene glycol | 10 | g |
| 2-pyrrolidone q.s. to | 100 | ml |

To prepare the formulation add the triclabendazole, propylene glycol and 2-pyrrolidone. Warm to 70° C. and stir to dissolve. Cool mix to less than 25° C. and dilute to volume with 2-pyrrolidone.

### Example 20

| Ingredients | Quantity | |
|---|---|---|
| Triclabendazole | 20 | g |
| Ethyl alcohol | 10 | g |
| 2-pyrrolidone q.s. to | 100 | ml |

To prepare the formulation add the triclabendazole, ethyl alcohol and 2-pyrrolidone. Warm to 70° C. and stir to dissolve. Cool mix to less than 25° C. and dilute to volume with 2-pyrrolidone.

### Example 21

| Ingredients | Quantity | |
|---|---|---|
| Triclabendazole | 10 | g |
| 2-pyrrolidone q.s. to | 100 | ml |

To prepare the formulation add the triclabendazole, and 2-pyrrolidone. Warm to 70° C. and stir to dissolve. Cool mix to less than 25° C. and dilute to volume with 2-pyrrolidone.

### Example 22

| Ingredients | Quantity |
|---|---|
| Triclabendazole | 10 g |
| NMP | 10 g |
| 2-pyrrolidone q.s. to | 100 ml |

To prepare the formulation add the triclabendazole, NMP and 2-pyrrolidone. Warm to 70° C. and stir to dissolve. Cool mix to less than 25° C. and dilute to volume with 2-pyrrolidone.

### Example 23

| Ingredients | Quantity |
|---|---|
| Triclabendazole | 10 g |
| Benzyl alcohol | 10 g |
| 2-pyrrolidone q.s. to | 100 ml |

To prepare the formulation add the triclabendazole, benzyl alcohol and 2-pyrrolidone. Warm to 70° C. and stir to dissolve. Cool mix to less than 25° C. and dilute to volume with 2-pyrrolidone.

### Example 24

| Ingredients | Quantity |
|---|---|
| Triclabendazole | 10 g |
| Glycerol formal | 10 g |
| 2-pyrrolidone q.s. to | 100 ml |

To prepare the formulation add the triclabendazole, glycerol formal and 2-pyrrolidone. Warm to 70° C. and stir to dissolve. Cool mix to less than 25° C. and dilute to volume with 2-pyrrolidone.

### Example 25

| Ingredients | Quantity |
|---|---|
| Triclabendazole | 10 g |
| Propylene glycol | 10 g |
| 2-pyrrolidone q.s. to | 100 ml |

To prepare the formulation add the triclabendazole, propylene glycol and 2-pyrrolidone. Warm to 70° C. and stir to dissolve. Cool mix to less than 25° C. and dilute to volume with 2-pyrrolidone.

### Example 26

| Ingredients | Quantity |
|---|---|
| Triclabendazole | 10 g |
| Ethyl alcohol | 10 g |
| 2-pyrrolidone q.s. to | 100 ml |

To prepare the formulation add the triclabendazole, ethyl alcohol and 2-pyrrolidone. Warm to 70° C. and stir to dissolve. Cool mix to less than 25° C. and dilute to volume with 2-pyrrolidone.

### Example 27

| Ingredients | Quantity |
|---|---|
| Triclabendazole | 5 g |
| 2-pyrrolidone q.s. to | 100 ml |

To prepare the formulation add the triclabendazole, and 2-pyrrolidone. Warm to 70° C. and stir to dissolve. Cool mix to less than 25° C. and dilute to volume with 2-pyrrolidone.

### Example 28

| Ingredients | Quantity |
|---|---|
| Triclabendazole | 5 g |
| NMP | 10 g |
| 2-pyrrolidone q.s. to | 100 ml |

To prepare the formulation add the triclabendazole, NMP and 2-pyrrolidone. Warm to 70° C. and stir to dissolve. Cool mix to less than 25° C. and dilute to volume with 2-pyrrolidone.

### Example 29

| Ingredients | Quantity |
|---|---|
| Triclabendazole | 5 g |
| Benzyl alcohol | 10 g |
| 2-pyrrolidone q.s. to | 100 ml |

To prepare the formulation add the triclabendazole, benzyl alcohol and 2-pyrrolidone. Warm to 70° C. and stir to dissolve. Cool mix to less than 25° C. and dilute to volume with 2-pyrrolidone.

### Example 30

| Ingredients | Quantity |
|---|---|
| Triclabendazole | 5 g |
| Glycerol formal | 10 g |
| 2-pyrrolidone q.s. to | 100 ml |

To prepare the formulation add the triclabendazole, glycerol formal and 2-pyrrolidone. Warm to 70° C. and stir to dissolve. Cool mix to less than 25° C. and dilute to volume with 2-pyrrolidone.

### Example 31

| Ingredients | Quantity |
|---|---|
| Triclabendazole | 5 g |
| Propylene glycol | 10 g |
| 2-pyrrolidone q.s. to | 100 ml |

To prepare the formulation add the triclabendazole, propylene glycol and 2-pyrrolidone. Warm to 70° C. and stir to dissolve. Cool mix to less than 25° C. and dilute to volume with 2-pyrrolidone.

### Example 32

| Ingredients | Quantity |
|---|---|
| Triclabendazole | 5 g |
| Ethyl alcohol | 10 g |
| 2-pyrrolidone q.s. to | 100 ml |

To prepare the formulation add the triclabendazole, ethyl alcohol and 2-pyrrolidone. Warm to 70° C. and stir to dissolve. Cool mix to less than 25° C. and dilute to volume with 2-pyrrolidone.

### Example 33

| Ingredients | Quantity |
|---|---|
| Triclabendazole | 40 g |
| PEG 400 q.s. to | 100 ml |

To prepare the formulation add the triclabendazole and PEG 400. Warm to 70° C. and stir to dissolve. Cool mix to less than 25° C. and dilute to volume with PEG 400.

### Example 34

| Ingredients | Quantity |
|---|---|
| Triclabendazole | 40 g |
| Glycerol formal | 10 g |
| PEG 400 q.s. to | 100 ml |

To prepare the formulation add the triclabendazole, glycerol formal and PEG 400. Warm to 70° C. and stir to dissolve. Cool mix to less than 25° C. and dilute to volume with PEG 400.

### Example 35

| Ingredients | Quantity |
|---|---|
| Triclabendazole | 20 g |
| PEG 400 q.s. to | 100 ml |

To prepare the formulation add the triclabendazole and PEG 400. Warm to 70° C. and stir to dissolve. Cool mix to less than 25° C. and dilute to volume with PEG 400.

### Example 36

| Ingredients | Quantity |
|---|---|
| Triclabendazole | 20 g |
| Glycerol formal | 10 g |
| PEG 400 q.s. to | 100 ml |

To prepare the formulation add the triclabendazole, glycerol formal and PEG 400. Warm to 70° C. and stir to dissolve. Cool mix to less than 25° C. and dilute to volume with PEG 400.

### Example 37

| Ingredients | Quantity | |
|---|---|---|
| Triclabendazole | 20 | g |
| Benzyl alcohol | 10 | g |
| PEG 400 q.s. to | 100 | ml |

To prepare the formulation add the triclabendazole, benzyl alcohol and PEG 400. Warm to 70° C. and stir to dissolve. Cool mix to less than 25° C. and dilute to volume with PEG 400.

### Example 38

| Ingredients | Quantity | |
|---|---|---|
| Triclabendazole | 20 | g |
| Propylene glycol | 10 | g |
| PEG 400 q.s. to | 100 | ml |

To prepare the formulation add the triclabendazole, propylene glycol and PEG 400. Warm to 70° C. and stir to dissolve. Cool mix to less than 25° C. and dilute to volume with PEG 400.

### Example 39

| Ingredients | Quantity | |
|---|---|---|
| Triclabendazole | 20 | g |
| Ethyl alcohol | 10 | g |
| PEG 400 q.s. to | 100 | ml |

To prepare the formulation add the triclabendazole, ethyl alcohol and PEG 400. Warm to 70° C. and stir to dissolve. Cool mix to less than 25° C. and dilute to volume with PEG 400.

### Example 40

| Ingredients | Quantity | |
|---|---|---|
| Triclabendazole | 10 | g |
| PEG 400 q.s. to | 100 | ml |

To prepare the formulation add the triclabendazole and PEG 400. Warm to 70° C. and stir to dissolve. Cool mix to less than 25° C. and dilute to volume with PEG 400.

### Example 41

| Ingredients | Quantity | |
|---|---|---|
| Triclabendazole | 10 | g |
| Glycerol formal | 10 | g |
| PEG 400 q.s. to | 100 | ml |

To prepare the formulation add the triclabendazole, glycerol formal and PEG 400. Warm to 70° C. and stir to dissolve. Cool mix to less than 25° C. and dilute to volume with PEG 400.

### Example 42

| Ingredients | Quantity | |
|---|---|---|
| Triclabendazole | 10 | g |
| Benzyl alcohol | 10 | g |
| PEG 400 q.s. to | 100 | ml |

To prepare the formulation add the triclabendazole, benzyl alcohol and PEG 400. Warm to 70° C. and stir to dissolve. Cool mix to less than 25° C. and dilute to volume with PEG 400.

### Example 43

| Ingredients | Quantity | |
|---|---|---|
| Triclabendazole | 10 | g |
| Propylene glycol | 10 | g |
| PEG 400 q.s. to | 100 | ml |

To prepare the formulation add the triclabendazole, propylene glycol and PEG 400. Warm to 70° C. and stir to dissolve. Cool mix to less than 25° C. and dilute to volume with PEG 400.

### Example 44

| Ingredients | Quantity | |
|---|---|---|
| Triclabendazole | 10 | g |
| Ethyl alcohol | 10 | g |
| PEG 400 q.s. to | 100 | ml |

To prepare the formulation add the triclabendazole, ethyl alcohol and PEG 400. Warm to 70° C. and stir to dissolve. Cool mix to less than 25° C. and dilute to volume with PEG 400.

### Example 45

| Ingredients | Quantity | |
|---|---|---|
| Triclabendazole | 5 | g |
| PEG 400 q.s. to | 100 | ml |

To prepare the formulation add the triclabendazole and PEG 400. Warm to 70° C. and stir to dissolve. Cool mix to less than 25° C. and dilute to volume with PEG 400.

### Example 46

| Ingredients | Quantity | |
|---|---|---|
| Triclabendazole | 5 | g |
| Glycerol formal | 10 | g |
| PEG 400 q.s. to | 100 | ml |

To prepare the formulation add the triclabendazole, glycerol formal and PEG 400. Warm to 70° C. and stir to dissolve. Cool mix to less than 25° C. and dilute to volume with PEG 400.

### Example 47

| Ingredients | Quantity | |
|---|---|---|
| Triclabendazole | 5 | g |
| Benzyl alcohol | 10 | g |
| PEG 400 q.s. to | 100 | ml |

To prepare the formulation add the triclabendazole, benzyl alcohol and PEG 400. Warm to 70° C. and stir to dissolve. Cool mix to less than 25° C. and dilute to volume with PEG 400.

### Example 48

| Ingredients | Quantity | |
|---|---|---|
| Triclabendazole | 5 | g |
| Propylene glycol | 10 | g |
| PEG 400 q.s. to | 100 | ml |

To prepare the formulation add the triclabendazole, propylene glycol and PEG 400. Warm to 70° C. and stir to dissolve. Cool mix to less than 25° C. and dilute to volume with PEG 400.

### Example 49

| Ingredients | Quantity | |
|---|---|---|
| Triclabendazole | 5 | g |
| Ethyl alcohol | 10 | g |
| PEG 400 q.s. to | 100 | ml |

To prepare the formulation add the triclabendazole, ethyl alcohol and PEG 400. Warm to 70° C. and stir to dissolve. Cool mix to less than 25° C. and dilute to volume with PEG 400.

It will be appreciated that the above formulations are examples only and that other formulations are contemplated wherein the solvent system may include any combination of 2-pyrrolidone and/or liquid polyethylene glycol with additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.

Furthermore, it will be appreciated that the formulation may further include additional anthelmintic actives or other medicaments which are soluble in the solvent system. For example, the additional anthelmintic active or actives may be selected from the group comprising avermectins, milbemycins, tetramisole and levamisole. Abamectin has been found to be particularly suitable.

The formulation may also further include excipients such as dyes, preservatives, stabilisers, buffers, thickeners, anti-foaming agents, spreading agents and the like.

### Stability Trials

Various formulations containing 2-pyrrolidone or PEG were prepared and stored at room temperature and refrigeration temperature for 30 days. The formulations showed no deposition at room temperature or under refrigeration.

### Efficacy Trials

To assess the field performance of the invention a study was undertaken to compare the bioavailability of a formulation of the present invention with that of a formulation of the commercially available triclabendazole pour-on formulation as described in PCT/NZ00/00053.

A high concentration 60% triclabendazole pour-on formulation was made as described in Example 4 (with the addition of abamectin) and as shown below (‘Formulation 1’).

Formulation 1

| Ingredient | G/100 ml |
|---|---|
| Abamectin | 1 |
| Triclabendazole | 60 |
| N-methyl pyrrolidone | 10 |
| 2-pyrrolidone | To volume |

The commercially available lower concentration 30% pour-on formulation as described in PCT/NZ00/00053 has the following formulation (‘Formulation 2’).

Formulation 2

| Ingredient | G/100 ml |
|---|---|
| Abamectin | 0.5 |
| Triclabendazole | 30 |
| Benzyl alcohol | 5 |
| Diethyl glycol monobutyl ether | 50 |
| Polyethylene glycol | To volume |

The trial included ten animals divided into two groups of five animals. At day zero of the trial all animals were treated. Group 1 animals were treated with Formulation 2 and Group 2 animals were treated with Formulation 1. Both formulations were applied topically to the skin surface of the animal. To minimise the possibility of licking, all animals were treated on a shaved portion of the neck and isolated in individual paddocks for the duration of the trial.

Blood samples were taken at days 4, 6, 8 and 10. Comparative levels of the active triclabendazole sulphoxide (which is the metabolite that appears in the body as the triclabendazole breaks down) were measured in the animals and the results are shown in the following table 5 (measurements of triclabendazole sulphoxide are expressed in ng/ml).

The study measured comparative levels of the metabolite triclabendazole sulphoxide component in animals expressed in ng/ml. (Triclabendazole breaks down to the sulphoxide and the sulphone in the body).

Both formulations were applied topically and to minimise the possibility of licking all animals were treated on a shaved portion of the neck and isolated in individual paddocks for the duration of the trial. Blood samples were taken at Days 4, 6, 8, and 10

The results of the study clearly demonstrated that at least in terms of levels of triclabendazole sulphoxide the 60% formulation was capable of delivering a result comparable to the commercial 30% formulation.

TABLE 2
Group 1 Commercially available Triclabendazole Pour-On

Cattle ID No.

| | 2 | 127 | 250 | 1022 | 1038 | Average |
|---|---|---|---|---|---|---|
| Day 0 | <LOD | <LOD | <LOD | <LOD | <LOD | |
| Day 4 | 0.75 | 0.59 | 0.42 | 0.91 | 0.81 | 0.696 |
| Day 6 | 0.41 | 0.42 | 0.33 | 0.42 | 0.59 | 0.434 |
| Day 8 | 0.27 | 0.23 | 0.27 | 0.29 | 0.48 | 0.308 |
| Day 10 | 0.53 | 0.16 | 0.22 | 0.29 | 0.44 | 0.328 |
| AUC* | 4.1 | 3.2 | 2.7 | 4.5 | 5 | 3.9 |
| (μg · d/mL) | | | | | | |

Group 2 60% Triclabendazole Pour-on Solution

Cattle ID No.

| | 238 | 422 | 445 | 1041 | 9980 | |
|---|---|---|---|---|---|---|
| Day 0 | <LOD | <LOD | <LOD | <LOD | <LOD | |
| Day 4 | 0.32 | 1.84 | 0.46 | 0.2 | 0.38 | 0.64 |
| Day 6 | 0.49 | 0.42 | 0.54 | 0.31 | 0.43 | 0.438 |
| Day 8 | 0.36 | 0.37 | 0.4 | 0.2 | 0.57 | 0.38 |
| Day 10 | 0.31 | 0.68 | 0.39 | 0.55 | 0.38 | 0.462 |
| AUC* | 3 | 7.8 | 3.6 | 2.2 | 3.5 | 4.02 |
| (μg · d/mL) | | | | | | |

*AUC—Area under concentration-time curve

The results of the study clearly demonstrate that at least in terms of levels of triclabendazole sulphoxide the formulation of the present invention is capable of delivering a result comparable to a commercially available formulation as described in PCT/NZ00/00053 which is known to provide effective treatment of parasitic infections, including fasciolisis.

### Advantages

The triclabendazole formulations of the present invention are advantageous as they allow the inclusion of triclabendazole in solution at significantly higher concentrations than previously known. This is advantageous as it allows the administration of an effective amount of triclabendazole in a low volume dose.

By way of example, pour-on formulations including triclabendazole in solution at about 40% w/v or greater will deliver an effective dose of triclabendazole to a 350 kg cow in about 25 ml of formulation. This smaller volume is particularly advantageous as it marries with the amount of liquid a pour-on gun can deliver. In addition, it increases the number of animals that may be treated out of the same pack thereby decreasing the need to change packs. The lower volume also further decreases the risk of the triclabendazole running off the back of the animal.

In field use these formulations would be highly desirable as they could provide the flexibility to deliver high concentrations of triclabendazole to an animal in a relatively small amount of solvent. Concentrated quantities of triclabendazole in solution could be delivered at a rate of as low as 1 ml per 20 kg of body weight, which equates to as little as 17.5 ml of formulation for a 350 kg cow.

### Variations

It is envisaged that the formulations of the invention may be modified by the inclusion of additional excipients without departing from the spirit and scope of the invention.

In particular it may be desirable to include dyes in the formulation to allow easy identification of treated animals. Alternately other excipients such as preservatives, stabilisers, buffers, thickeners, anti-foaming agents, spreading agents and the like may be included to modify the formulation to specific animals.

Also, additional excipients may be included within the formulations of the present invention to tailor them to suit a specific administration method.

In this specification the words “includes”, “including” and the like and “comprises”, “comprising” and the like should be considered synonymous and be given a non-exhaustive meaning.

Finally it will be appreciated that various other alterations and modifications may be made to the foregoing without departing from the scope of the invention.

The invention is further described by the following numbered paragraphs:
1. A veterinary anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.
2. A veterinary anthelmintic formulation of paragraph 1, wherein the solvent system also includes one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.
3. A stable liquid veterinary pour-on anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.
4. A pour-on formulation of paragraph 3, wherein the solvent system also includes one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.
5. A pour-on formulation of paragraph 4, wherein the solvent system contains 2-pyrrolidone and at least one additional solvent selected from the group comprising butyl dioxitol, N-methyl pyrrolidone, benzyl alcohol, glycerol formal, propylene glycol and ethyl alcohol.
6. A pour-on formulation of any one of paragraphs 3 to 5, wherein the triclabendazole is present in the range of 5-60% w/v.
7. A pour-on formulation of any one of paragraphs 3 to 6, wherein the triclabendazole is present in a sufficiently high concentration such that an effective amount of triclabendazole may be delivered to an animal in a volume of substantially 25 ml or less.
8. A pour-on formulation of any one of paragraphs 3 to 7, wherein the triclabendazole is present in the range of 40-60% w/v.
9. A pour-on formulation of any previous paragraph, wherein the formulation includes at least one additional anthelmintic active or other medicament which is soluble in the solvent system.
10. A pour-on formulation of paragraph 9, wherein the additional anthelmintic active or actives are selected from the group comprising avermectins, milbemycins, tetramisole and levamisole.
11. A pour-on formulation of paragraph 9 or paragraph 10, wherein the additional anthelmintic active is an avermectin, such as abamectin.
12. A pour-on formulation of any previous paragraph wherein the formulation further includes excipients such as dyes, preservatives, stabilisers, buffers, thickeners, anti-foaming agents, spreading agents and the like.
13. A pour-on formulation of any previous paragraph wherein the formulation contains (a) about 60% w/v of triclabendazole, (b) about 1.0% w/v of abamectin, and a solvent system, wherein the solvent system includes N-methyl pyrrolidone in an amount of about 10% w/v and 2-pyrrolidone in an amount of about 29% w/v.
14. A method of treating parasites including fasciolisis in warm blooded animals by administering to an animal a formulation of any one of paragraphs 1 to 13.
15. A method of treating parasites including fasciolisis in warm blooded animals by providing a formulation of any one of paragraphs 1 to 13 in the form of a pour-on formulation and administering a volume of substantially 25 ml or less of the pour-on formulation on to the skin surface of an animal.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A veterinary anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.

2. A veterinary anthelmintic formulation as claimed in claim 1, wherein the solvent system also includes one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol.

3. A stable liquid veterinary pour-on anthelmintic formulation containing a stable solution of triclabendazole, wherein the solution contains an effective amount of triclabendazole and a solvent system containing one or more solvents, wherein at least one of the solvents is selected from the group comprising 2-pyrrolidone and liquid polyethylene glycol.

4. A pour-on formulation as claimed in claim 3,
wherein the solvent system also includes one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol or
wherein the solvent system also includes one or more additional solvents selected from the group comprising benzyl alcohol, propylene glycol, N-methyl pyrrolidone, glycerol formal, glycol ethers, butyl dioxitol, methyl alcohol, ethyl alcohol, isopropyl alcohol, 1-butanol and 1-hexanol and
wherein the solvent system contains 2-pyrrolidone and at least one additional solvent selected from the group comprising butyl dioxitol, N-methyl pyrrolidone, benzyl alcohol, glycerol formal, propylene glycol and ethyl alcohol or
wherein the triclabendazole is present in the range of 5-60% w/v or
wherein the triclabendazole is present in a sufficiently high concentration such that an effective amount of triclabendazole may be delivered to an animal in a volume of substantially 25 ml or less or
wherein the triclabendazole is present in the range of 40-60% w/v.

5. A pour-on formulation as claimed in claim 1, wherein the formulation includes at least one additional anthelmintic active or other medicament which is soluble in the solvent system.

6. A pour-on formulation as claimed in claim 5,
wherein the additional anthelmintic active or actives are selected from the group comprising avermectins, milbemycins, tetramisole and levamisole or
wherein the additional anthelmintic active is an avermectin, such as abamectin.

7. A pour-on formulation as claimed in claim 3, wherein the formulation includes at least one additional anthelmintic active or other medicament which is soluble in the solvent system.

8. A pour-on formulation as claimed in claim 7,
wherein the additional anthelmintic active or actives are selected from the group comprising avermectins, milbemycins, tetramisole and levamisole or
wherein the additional anthelmintic active is an avermectin, such as abamectin.

9. A pour-on formulation as claimed in claim 1
wherein the formulation further includes excipients such as dyes, preservatives, stabilisers, buffers, thickeners, anti-foaming agents, spreading agents and the like or
wherein the formulation contains (a) about 60% w/v of triclabendazole, (b) about 1.0% w/v of abamectin, and a solvent system, wherein the solvent system includes N-methyl pyrrolidone in an amount of about 10% w/v and 2-pyrrolidone in an amount of about 29% w/v.

10. A pour-on formulation as claimed in claim 3
wherein the formulation further includes excipients such as dyes, preservatives, stabilisers, buffers, thickeners, anti-foaming agents, spreading agents and the like or
wherein the formulation contains (a) about 60% w/v of triclabendazole, (b) about 1.0% w/v of abamectin, and a solvent system, wherein the solvent system includes N-methyl pyrrolidone in an amount of about 10% w/v and 2-pyrrolidone in an amount of about 29% w/v.

11. A method of treating parasites including fasciolisis in warm blooded animals by administering to an animal a formulation as claimed in claim 1.

12. A method of treating parasites including fasciolisis in warm blooded animals by providing a formulation as claimed in claim 1 in the form of a pour-on formulation and administering a volume of substantially 25 ml or less of the pour-on formulation on to the skin surface of an animal.

13. A method of treating fasciolisis in warm blooded animals by providing a formulation as claimed in claim 1 in the form of a pour-on formulation and administering a volume capable of delivering at least 1 mg of triclabendazole per 10 kg of body weight of the animal to be treated.

14. A method of treating parasites including fasciolisis in warm blooded animals by administering to an animal a formulation as claimed in claim 3.

15. A method of treating parasites including fasciolisis in warm blooded animals by providing a formulation as claimed in claim 3 in the form of a pour-on formulation and administering a volume of substantially 25 ml or less of the pour-on formulation on to the skin surface of an animal.

16. A method of treating fasciolisis in warm blooded animals by providing a formulation as claimed in claim 3 in the form of a pour-on formulation and administering a volume capable of delivering at least 1 mg of triclabendazole per 10 kg of body weight of the animal to be treated.
